(19) United States
(12) Patent Application Publication
(10) Pub. No.: US 2004/0266797 A1
(43) Pub. Date: Dec. 30, 2004

(54) Use of potent,selective and non toxic c-kit inhibitors for treating tumor angiogensis

(76) Inventors: Alain Moussy, Paris (FR); Jean-Pierre Kinet, Lexington, MA (US)

Correspondence Address: SUGHRUE MION, PLLC, 2100 PENNSYLVANIA AVENUE, N.W., SUITE 800, WASHINGTON, DC, 20037, US

(21) Appl. No.: 10/482,177

(22) PCT Filed: Jun. 28, 2002
(86) PCT No.: PCT/IB02/03295

Related U.S. Application Data
(60) Provisional application No. 60/301,407, filed on Jun. 29, 2001.

(51) Int. Cl. A61K 31/517; A61K 31/505

(52) U.S. Cl. 514/266.4; 514/275; 514/265.1; 514/249; 514/312; 514/352; 514/406; 514/419

(57) ABSTRACT

The present invention relates to a method for inhibiting tumor angiogenesis comprising administering a c-kit inhibitor to a human in need of such treatment, more particularly a non toxic, potent and selective c-kit inhibitor, wherein said inhibitor is unable to promote death of IL-3 dependent cells cultured in presence of IL-3.

The present invention relates to a method for inhibiting tumor angiogenesis comprising administering a c-kit inhibitor to a human in need of such treatment, more particularly a non toxic, potent and selective c-kit inhibitor, wherein said inhibitor is unable to promote death of IL-3 dependent cells cultured in presence of IL-3.

In 1971, Folkman J. (Tumor angiogenesis: Therapeutic implications., N. Engl. Jour. Med. 285:1182-1186) postulated that every increase in tumor cell population must be preceded by an increase in new capillaries converging on the tumor. Since, many evidence have accumulated demonstrating that the growth of new blood vessels from a preexisting microvascular bed is necessary for the growth, maintenance, and metastasis of solid tumors.

Different compounds are being tried out for their potential therapeutic application in tumor angiogenesis. Among these compounds, Marimastat (British Biotech) and BMS-275291 (Bristol-Myers Squibb) are synthetic inhibitors of matrix metalloproteinases (MMPs), Neovastat (Aeterna) is a naturally occurring MMP inhibitor, Squalamine (Magainin Pharmaceuticals) is extracted from dogfish shark liver, Endostatin (EntreMed) is an inhibitor of endothelial cells growth, SU5416 and SU6668 (Sugen) block VEGF/PDGF receptor signaling.

While these compounds block a particular stimulus leading to angiogenesis, they don't abolish all the pathways involved in the induction of new blood vessels, which results from the concomitant action of several growth factors and cytokines. These signals leading to tumor angiogenesis depend on the interaction of different tumor components tumor parenchymal cells, endothelial cells, infiltrating cells from the bloodstream, and mast cells.

In connection with the present invention, we have determined that mast cells are in fact a major player in tumor angiogenesis due to their ability to secrete numerous growth factors and cytokines that ultimately balance the equilibrium in favor of vascular endothelial cells growth.

Mast cells (MC) are tissue elements derived from a particular subset of hematopoietic stem cells that express CD34, c-kit and CD13 antigens (Kirshenbaum et al, Blood. 94: 2333-2342, 1999 and Ishizaka et al, Curr Opin Immunol. 5: 937-43, 1993). Immature MC progenitors circulate in the bloodstream and differentiate in tissues. These differentiation and proliferation processes are under the influence of cytokines, one of utmost importance being Stem Cell Factor (SCF), also termed Kit ligand (KL), Steel factor (SL) or Mast Cell Growth Factor (MCGF). SCF receptor is encoded by the protooncogene c-kit, that belongs to type III receptor tyrosine kinase subfamily (Boissan and Arock, J Leukoc Biol. 67: 135-48, 2000). This receptor is also expressed on others hematopoietic or non hematopoietic cells. Ligation of c-kit receptor by SCF induces its dimerization followed by its transphosphorylation, leading to the recruitement and activation of various intracytoplasmic substrates. These activated substrates induce multiple intracellular signaling pathways responsible for cell proliferation and activation (Boissan and Arock, 2000). Mast cells are characterized by their heterogeneity, not only regarding tissue location and structure but also at the functional and histochemical levels (Aldenborg and Enerback., Histochem. J. 26: 587-96, 1994 ; Bradding et al. J Immunol. 155: 297-307, 1995; Irani et al, J Immunol. 147: 247-53, 1991; Miller et al, Curr Opin Immunol. 1: 637-42, 1989 and Welle et al, J Leukoc Biol. 61: 233-45, 1997).

Several observations have suggested the implication of mast cells in the pathogenesis of cancer and angiogenesis. First, mast cells have been shown to accumulate within and around solid tumors (Fisher E. and Fisher B. Role of mast cells in tumor growth. Arch. Pathol., 79: 185-191, 1965). Second, mast cells are distributed along blood vessels (Eady R. et al, Mast cell population density, blood vessel density and histamine content in normal skin. Br. J. Dermatol., 100: 635-640, 1979). Mast cell degranulation induces neovascularization in rat mesentery (Norrby K. et al, Mast-cell-mediated angiogenesis: a novel experimental model using the rat mesentery. Virchows Arch. B Cell Pathol. Incl. Mol. Pathol., 52: 195-206, 1986) and in the chick chorioallantoic membrane (Clinton M. et al. Effect of the mast cell activator compound 48/80 and heparin on angiogenesis in the chick chornoallantoic membrane. Int. J. Microcirc. Clin. Exp., 7: 315-326, 1988.).

Furthermore, when tumor cells are injected into a chick embryo, a 40-fold increase in mast cell density has been observed around the tumor implantation site compared with normal tissue (Kessler D. and Folkman J. Mast cells and tumor angiogenesis. Int. J. Cancer, 18: 703-709, 1976.). Injection of mast cell suspensions into animals induce an acceleration of tumor growth (Roche W., The nature and significance of tumor-associated mast cells. J. Pathol., 148: 175-182, 1986.), whereas decreasing the number of tissue mast cells leads to depression of tumor growth (Scott K.,. The mast cell, its amines, and tumor growth in rodents and man. Ann. NY Acad. Sci., 103: 285-312, 1963).

In addition, inhibiting mast cell degranulation with disodium cromoglycate has been demonstrated to significantly depresse tumor growth (Ionov I., Inhibition of mast cell activity as a new approach to anticancer therapy. Int. J. Radiat. Biol., 60: 287-291, 1991). More recently, it has been suggested that mast cells in tumors modulate the neovascularization process (Wei Zhang et al, Modulation of Tumor Angiogenesis by Stem Cell Factor, Cancer Research 60, 6757-6762, Dec. 1, 2000).

The present invention goes further based in the fact that tumor cell lines express stem cell factor SCF and display c-kit receptors (Turner et al, , Blood Volume 80, Issue 2, pp. 374-381, 1992). It is proposed here that tumor cells activate mast cells proliferation via SCF, which in turn degranulate and release mediators such as histamine, TNF, IL-8, VEGF or bFGF that acts together to promote angiogenesis. While blood vessels develop, tumor is allowed to grow bigger, which results in an increase of SCF release. Consequently, an activating feedback loop is created ultimately leading to further activation of mast cells as well as growth of tumors and metastasis.

In addition, the role of mast cells in the process of tumor angiogenesis was confirmed by comparing the rates of tumor vascularization, growth and metastasis in control WBB6F1(−)+/+ mice and in their mast-cell- deficient WBB6F1-W/Wv littermates injected with MB49 murine bladder carcinoma cells. The results of these experiments demonstrated that in mast-cell-deficient mice injected with tumor cells, there is a decreased number of capillaries at the tumor periphery, reduced tumor size relative to control mice, and an absence of metastases. These results have also shown that the reduction of blood vessels at the tumor periphery might lead to a reduction in the number of metastatic cells in mast-cell- deficient mice.

The relevance of the above mentioned hypothesis towards the human situation has been confirmed by studies conducted in patients suffering from lung cancer, in whom mast cell counts were significantly higher than in control normal tissues. Good correlation was observed between intratumoral mast cell counts and microvessel counts. Double staining showed highly angiogenic areas densely populated with mast cells. Importantly, members in the high mast cell count group had significantly worse prognosis than those in the low mast cell count group.

From these studies confirming a mutual activation between tumor cells and mast cells, we can conclude that tumor-released vascular endothelial growth factors is related to mast cell accumulation, that intratumoral mast cells produce angiogenic factors, and that stromal mast cells correlate with angiogenesis and poor outcome in lung cancer.

In this regard, the general aim of the invention is to provide therapeutic strategies aiming at blocking the activation and the survival of mast cells which are involved in tumor angiogenesis. This can be done by any means leading to mast cells death or inactivation. For example, it has been found that targeting c-kit or c-kit signaling is particularly suited to reach this goal. To this end, tyrosine kinase inhibitors that are non toxic and specific for mast cells are contemplated. These inhibitors are unable to promote death of IL-3 dependent cells cultured in presence of IL-3. Among such inhibitors, c-kit specific kinase inhibitors to inhibit mast cell proliferation, survival and activation are of a particular interest for clinical uses.

### DESCRIPTION

Therefore, the present invention relates to a method for treating tumor angiogenesis comprising administering a tyrosine kinase inhibitor to a mammalian in need of such treatment, wherein said inhibitor is unable to promote death of IL-3 dependent cells cultured in presence of IL-3.

Tyrosine kinase inhibitors are selected for example from bis monocyclic, bicyclic or heterocyclic aryl compounds (WO 92/20642), vinylene-azaindole derivatives (WO 94/14808) and 1-cycloproppyl-4-pyridyl-quinolones (U.S. Pat. No. 5,330,992), Styryl compounds (U.S. Pat. No. 5,217,999), styryl-substituted pyridyl compounds (U.S. Pat. No. 5,302,606), seleoindoles and selenides (WO 94/03427), tricyclic polyhydroxylic compounds (WO 92/21660) and benzylphosphonic acid compounds (WO 91/15495), pyrimidine derivatives (U.S. Pat. No. 5,521,184 and WO 99/03854), indolinone derivatives and pyrrol-substituted indolinones (U.S. Pat. No. 5,792,783, EP 934 931, U.S. Pat. No. 5,834,504, U.S. Pat. No. 5,883,116, , U.S. Pat. No. 5,883,113, U.S. Pat. No. 5,886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. No. 3,772,295 and U.S. Pat. No. 4,343,940) and aryl and heteroaryl quinazoline (U.S. Pat. No. 5,721,237, U.S. Pat. No. 5,714,493, U.S. Pat. No. 5,710,158 and WO 95/15758).

Preferably, said tyrosine kinase inhibitor is a non-toxic, selective and potent c-kit inhibitor. Such inhibitors can be selected from pyrimidine derivatives such as N-phenyl-2-pyrimidine-amine derivatives (U.S. Pat. No. 5,521,184 and WO 99/03854), indolinone derivatives and pyrrol-substituted indolinones (U.S. Pat. No. 5,792,783, EP 934 931, U.S. Pat. No. 5,834,504), U.S. Pat. No. 5,883,116, U.S. Pat. No. 5,883,113, U.S. Pat. No. 5, 886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. No. 3,772,295 and U.S. Pat. No. 4,343,940), 4-amino-substituted quinazolines (U.S. Pat. No. 3,470,182), 4-thienyl-2-(1H)-quinazolones, 6,7-dialkoxyquinazolines (U.S. Pat. No. 3,800,039), aryl and heteroaryl quinazoline (U.S. Pat. No. 5,721,237, U.S. Pat. No. 5,714,493, U.S. Pat. No. 5,710,158 and WO 95/15758), 4-anilinoquinazoline compounds (U.S. Pat. No. 4,464,375), and 4-thienyl-2-(1H)-quinazolones (U.S. Pat. No. 3,551,427).

So, preferably, the invention relates to a method for preventing or treating tumor angiogenesis comprising administering a non toxic, potent and selective c-kit inhibitor. Such inhibitor can be selected from pyrimidine derivatives, more particularly N-phenyl-2-pyrimidine-amine derivatives of formula I:

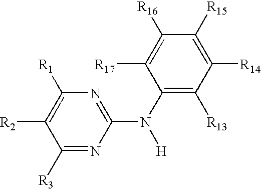

wherein the R1, R2, R3, R13 to R17 groups have the meanings depicted in EP 564 409 B 1, incorporated herein in the description.

Preferably, the N-phenyl-2-pyrimidine-amine derivative is selected from the compounds corresponding to formula II:

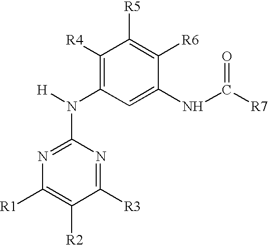

Wherein R1, R2 and R3 are independently chosen from H, F, Cl, Br, I, a C1-C5 alkyl or a cyclic or heterocyclic group, especially a pyridyl group;

R4, R5 and R6 are independently chosen from H, F, Cl, Br, I, a C1-C5 alkyl, especially a methyl group;

and R7 is a phenyl group bearing at least one substituent, which in turn possesses at least one basic site, such as an amino function.

Preferably, R7 is the following group:

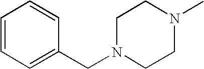

Among these compounds, the preferred are defined as follows:

R1 is a heterocyclic group, especially a pyridyl group,

R2 and R3 are H,

R4 is a C1-C3 alkyl, especially a methyl group,

R5 and R6 are H,

and R7 is a phenyl group bearing at least one substituent, which in turn possesses at least one basic site, such as an amino function, for example the group:

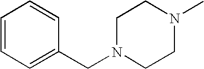

Therefore, in a preferred embodiment, the invention relates to a method for treating tumor angiogenesis comprising the administration of an effective amount of the compound known in the art as CGP57148B:

4-(4-méhylpipérazine-1-ylméthyl)-N-[4-méthyl-3-(4-pyridine-3-yl)pyrimidine-2 ylamino)phényl]-benzamide corresponding to the following formula:

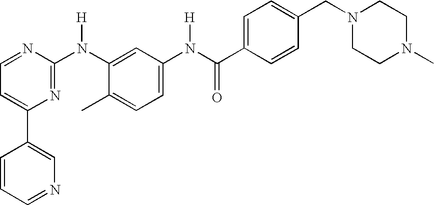

The preparation of this compound is described in example 21 of EP 564 409 and the β-form, which is particularly useful is described in WO 99/03854.

Alternatively, the invention relates to a method for treating tumor angiogenesis comprising administering a non toxic, potent and selective c-kit inhibitor to a mammalian in need of such treatment, selected from the group consisting of

indolinone derivatives, more particularly pyrrol-substituted indolinones,

monocyclic, bicyclic aryl and heteroaryl compounds, quinazoline derivatives,

and quinaxolines, such as 2-phényl-quinaxoline derivatives, for example 2-phenyl-6,7-dimethoxy quinaxoline.

Preferably, said inhibitors are unable to promote death of IL-3 dependent cells cultured in presence of IL-3.

In another embodiment, c-kit inhibitors as mentioned above are inhibitors of activated c-kit. In frame with the invention, the expression “activated c-kit” means a constitutively activated-mutant c-kit including at least one mutation selected from point mutations, deletions, insertions, but also modifications and alterations of the natural c-kit sequence (SEQ ID N^{o}1). Such mutations, deletions, insertions, modifications and alterations can occur in the transphosphorylase domain, in the juxtamembrane domain as well as in any domain directly or indirectly responsible for c-kit activity. The expression “activated c-kit” also means herein SCF-activated c-kit. Preferred and optimal SCF concentrations for activating c-kit are comprised between 5.10⁻⁷ M and 5.10⁻⁶ M, preferably around 2.10⁻⁶ M. In a preferred embodiment, the activated-mutant c-kit in step a) has at least one mutation proximal to Y823, more particularly between amino acids 800 to 850 of SEQ ID No 1 involved in c-kit autophosphorylation, notably the D816V, D816Y, D816F and D820G mutants. In another preferred embodiment, the activated-mutant c-kit in step a) has a deletion in the juxtamembrane domain of c-kit. Such a deletion is for example between codon 573 and 579 called c-kit d(573-579). The point mutation V559G proximal to the juxtamembrane domain c-kit is also of interest.

In this regard, the invention contemplates a method for treating tumor angiogenesis comprising administering to a mammalian in need of such treatment a compound that is a selective, potent and non toxic inhibitor of activated c-kit obtainable by a screening method which comprises:

a) bringing into contact (i) activated c-kit and (ii) at least one compound to be tested; under conditions allowing the components (i) and (ii) to form a complex,

b) selecting compounds that inhibit activated c-kit,

c) testing and selecting a subset of compounds identified in step b), which are unable to promote death of IL-3 dependent cells cultured in presence of IL-3.

This screening method can further comprise the step consisting of testing and selecting a subset of compounds identified in step b) that are inhibitors of mutant activated c-kit (for example in the transphosphorylase domain), which are also capable of inhibiting SCF-activated c-kit wild.

Alternatively, in step a) activated c-kit is SCF-activated c-kit wild.

A best mode for practicing this method consists of testing putative inhibitors at a concentration above 10 μM in step a). Relevant concentrations are for example 10, 15, 20, 25, 30, 35 or 40 μM.

In step c), IL-3 is preferably present in the culture media of IL-3 dependent cells at a concentration comprised between 0.5 and 10 ng/ml, preferably between 1 to 5 ng/ml.

Examples of IL-3 dependent cells include but are not limited to:

cell lines naturally expressing and depending on c-kit for growth and survival. Among such cells, human mast cell lines can be established using the following procedures normal human mast cells can be infected by retroviral vectors containing sequences coding for a mutant c-kit comprising the c-kit signal peptide and a TAOG sequence allowing to differentiate mutant c-kits from c-kit wild expressed in hematopoetic cells by means of antibodies.

This technique is advantageous because it does not induce cellular mortality and the genetic transfer is stable and gives satisfactory yields (around 20%). Pure normal human mast cells can be routinely obtained by culturing precursor cells originating from blood obtained from human umbilical vein. In this regard, heparinated blood from umbilical vein is centrifuged on a Ficoll gradient so as to isolate moaonucleated cells from other blood components. CD34+ precursor cells are then purified from the isolated cells mentioned above using the immunomagnetic selection system MACS (Miltenyi biotech). CD34+ cells are then cultured at 37° C. in 5% CO₂ atmosphere at a concentration of 10⁵ cells per ml in the medium MCCM (α-MEM supplemented with L-glutamine, penicillin, streptomycin, 5⁻⁵ M β-mercaptoethanol, 20% veal foetal serum, 1% bovine albumin serum and 100 ng/ml recombinant human SCF. The medium is changed every 5 to 7 days. The percentage of mast cells present in the culture is assessed each week, using May-Grünwal Giemsa or Toluidine blue coloration. Anti-tryptase antibodies can also be used to detect mast cells in culture. After 10 weeks of culture, a pure cellular population of mast cells (<98%) is obtained.

It is possible using standard procedures to prepare vectors expressing c-kit for transfecting the cell lines established as mentioned above. The cDNA of human c-kit has been described in Yarden et al., (1987) EMBO J.6 (11), 3341-3351. The coding part of c-kit (3000 bp) can be amplified by PCR and cloned, using the following oligonucleotides:

| | |
|---|---|
| 5′AAGAAGAGATGGTACCTCGAGGGGTGACCC3′ | (SEQ ID No2) |
| sens | |
| 5′CTGCTTCGCGGCCGCGTTAACTCTTCTCAACCA3′ | (SEQ ID No3) |

antisens

The PCR products, digested with NotI and XhoI, has been inserted using T4 ligase in the pFlag-CMV vector (SIGMA), which vector is digested with NotI and XhoI and dephosphorylated using CIP (Biolabs). The pFlag-CMV-c-kit is used to transform bacterial clone XL1-blue. The transformation of clones is verified using the following primers:

| | | |
|---|---|---|
| 5′AGCTCGTTTAGTGAACCGTC3′ | (SEQ ID No4) | sens, |
| 5′GTCAGACAAAATGATGCAAC3′ | (SEQ ID No5) | antisens. |

Directed mutagenesis is performed using relevant cassettes is performed with routine and common procedure known in the art.

The vector Migr-1 (ABC) can be used as a basis for constructing retroviral vectors used for transfecting mature mast cells. This vector is advantageous because it contains the sequence coding for GFP at the 3′ and of an IRES. These features allow to select cells infected by the retrovirus using direct analysis with a fluorocytometer. As mentioned above, the N-terminal sequence of c-kit c-DNA can be modified so as to introduce a Flag sequence that will be useful to discriminating heterogeneous from endogenous c-kit.

Other IL-3 dependent cell lines that can be used include but are not limited to:

BaF3 mouse cells expressing wild-type or mutated form of c-kit (in the juxtamembrane and in the catalytic sites) are described in Kitayama et al, (1996), Blood 88, 995-1004 and Tsujimura et al, (1999), Blood 93, 1319-1329.

IC-2 mouse cells expressing either c-kit^{WT} or c-kit^{D814Y} are presented in Piao et al, (1996), Proc. Natl. Acad. Sci. USA 93, 14665-14669.

IL-3 independent cell lines are:

HMC-1, a factor-independent cell line derived from a patient with mast cell leukemia, expresses a juxtamembrane mutant c-kit polypeptide that has constitutive kinase activity (Furitsu T et al, J Clin Invest. 1993;92:1736-1744 ; Butterfield et al, Establishment of an immature mast cell line from a patient with mast cell leukemia. Leuk Res. 1988;12:345-355 and Nagata et al, Proc Natl Acad Sci USA. 1995;92:10560-10564).

P815 cell line (mastocytoma naturally expressing c-kit mutation at the 814 position) has been described in Tsujimura et al, (1994), Blood 83, 2619-2626.

The extent to which component (ii) inhibits activated c-kit can be measured in vitro or in vivo. In case it is measured in vivo, cell lines expressing an activated-mutant c-kit, which has at least one mutation proximal to Y823, more particularly between amino acids 800 to 850 of SEQ ID No1 involved in c-kit autophosphorylation, notably the D816V, D816Y, D816F and D820G mutants, are preferred.

Example of cell lines expressing an activated-mutant c-kit are as mentioned above.

In another preferred embodiment, the method further comprises the step consisting of testing and selecting compounds capable of inhibiting c-kit wild at concentration below 1 μM. This can be measured in vitro or in vivo.

Therefore, compounds are identified and selected according to the method described above are potent, selective and non-toxic c-kit wild inhibitors.

Alternatively, the screening method according to the invention can be practiced in vitro In this regard, the inhibition of mutant-activated c-kit and/or c-kit wild can be measured using standard biochemical techniques such as immunoprecipitation and western blot. Preferably, the amount of c-kit phosphorylation is measured.

In a still further embodiment, the invention contemplates a method for treating tumor angiogenesis as depicted above wherein the screening comprises:

a) performing a proliferation assay with cells expressing a mutant c-kit (for example in the transphosphorylase domain), which mutant is a permanent activated c-kit, with a plurality of test compounds to identify a subset of candidate compounds targeting activated c-kit, each having an IC50<10 μM, by measuring the extent of cell death,

b) performing a proliferation assay with cells expressing c-kit wild said subset of candidate compounds identified in step (a), said cells being IL-3 dependent cells cultured in presence of IL-3, to identify a subset of candidate compounds targeting specifically c-kit,

c) performing a proliferation assay with cells expressing c-kit, with the subset of compounds identified in step b) and selecting a subset of candidate compounds targeting c-kit wild, each having an IC50<10 μM, preferably an IC50<1 μM, by measuring the extent of cell death.

Here, the extent of cell death can be measured by 3H thymidine incorporation, the trypan blue exclusion method or flow cytometry with propidium iodide. These are common techniques routinely practiced in the art.

Therefore, the invention embraces the use of the compounds defined above to manufacture a medicament for treating tumor angiogenesis in human.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions.

The pharmaceutical composition may be provided as a salt and can be formed with many acids, including but not limited to, hydrochloric, sulfuric, acetic, lactic, tartaric, malic, and succine, acids, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

Pharmaceutical compositions suitable for use in the invention include compositions wherein c-kit inhibitors are contained in an effective amount to achieve the intended purpose. The determination of an effective dose is well within the capability of those skilled in the art. A therapeutically effective dose refers to that amount of active ingredient, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio of toxic to therpeutic effects is the therapeutic index, and it can be expressed as the ratio, LD50/ED50. Pharmaceutical compositions which exhibit large therapeutic indices are preferred. As mentioned above, a tyrosine kinase inhibitor and more particularly a c-kit inhibitor according to the invention is unable to promote death of IL-3 dependent cells cultured in presence of IL-3.

Therefore, the invention is also aimed at the use of a non toxic, potent and selective c-kit inhibitor for preparing a medicament for treating tumor angiogenesis in human, more particularly the use of a tyrosine kinase inhibitor or a c-kit inhibitor as defined above as being unable to promote death of IL-3 dependent cells cultured in presence of IL-3 for the manufacture of a medicament for treating tumor angiogenesis.

Utility of the invention will further ensue from the detailed description below.

### EXAMPLE 1

### Identification of Pro-Angiogenic Genes Over-Expressed in Mast Cells

Genes expressed in mast cells, which contribute to the pathogenesis of diseases have been searched for. The purpose was to identify 1) genes expressed in different type of mast cells involved in different forms of mastocytosis and caused by mutations on the c-kit receptor and 2) genes expressed in mast cells involved in different pathologies, especially in the development of solid tumors, in metatasis as well as in inflammatory syndromes.

In a first approach, genes whose expression is linked to the differentiation of mast cells were identified from totipotent CD34+ cells, immature hematopoietic cells in course of differentiation and normal mature mast cells.

Partial cDNA Expression Arrays

Expression profile of CD34+ cells extracted from human bone marrow and expression profile of mature mast cells derived from these CD 34+ induced by Stem Cell Factor (SCF) were obtained and analyzed by the Atlas Software.

Two types of membranes were used. The first one allows to detect 588 genes which are <<general>> and the other one allows to detect genes belonging to the haematology domain.

Genes whose expression is significantly increased (≧×3) during mast cells differentiation are shown in the Table I below:

TABLE 1
PARTIAL TRANSCRIPTOME OF MAST CELLS
Membrane << general >>

| | | | Genes over-expressed in mast cells versus CD34+ |
|---|---|---|---|
| code | Ratio | Diff | cells Protein/gene |
| F3j | 4.008417 | 26450 | transcription factor ETR103; early growth |
| | | | response protein 1 (EGR-1) (KROX24); zinc |
| | | | finger protein 225 (AT225) |
| C3i | 4.694429 | 27590 | Notch4 |
| E2d | 5.850825 | 16454 | TIMP-3; mitogen-inducible gene 5 (mig-5) |
| C3j | 6.289161 | 16005 | Jagged 1 |
| F6e | 6.525074 | 14984 | platelet-derived growth factor A chain |
| | | | (PDGF-A) |
| B6b | 7.194957 | 30219 | C-kei |
| B6-c | 7.476532 | 26217 | proto-oncogene c-src1 tyrosine kinase |
| | | | domain |
| E1f | 9.513902 | 18986 | MMP-9; gelatinase B |
| D6h | 10.915376 | 16638 | LAR |
| B6a | 11.612540 | 13202 | C-fos |
| E1n | 23.914141 | 13611 | MMP-17 (MT4-MMP) |

Membrane <<Haematology>>

| Up regulated genes cut <3 | |
|---|---|
| Monocyte chemotactic factor | 38,9 |
| IL1 receptor antagonist | 33,9 |
| DNA binding protein inhibitor | 27,5 |
| CD9 antigen; p24; MIC3 | 11,3 |
| RGS1 B-cell activation prot | 11,7 |
| LIF; differentiation-stimulating | 10,1 |
| factor | |
| ICAM1; CD 54 antigen | 9,4 |
| ST2 protein precursor | 7,9 |
| GATA2 | 4,7 |
| BTK | 4,1 |
| JAK3 | 3,8 |
| CD44 precursor | 3,5 |

Over-Expression Notch4 and Jagged 1

Differentiation of CD 43+ in mast cells results in a concomitant increase of the expression of Notch4 and its ligand Jagged 1.

Notch 4 is a membrane receptor present in embryonic cells and in the endothelium. Jagged and notch4 are involved in the mechanism leading to angiogenesis. Notch signaling can regulate the angiogenic process since Notch4/int-3 and Jagged-1 are able to induce cultured endothelial cells to form cellular structures with morphological and biochemical properties of endothelial microvessels; Uyttendaele H. et al, Notch4/int-3, a mammary proto-oncogene, is an endothelial cell-specific Notch gene, Development. 122: 2251-59 (1996) and Uyttendaele, H. et al (2000) Notch4 and Jagged1 induce microvessel differentiation of rat brain endothelial cells. Microvascular Res. Volkhard L. et al, (Am J Pathol 2001, 159:875-883) also reported that Jagged regulation of cell-cell and cell-matrix interactions may contribute to the control of cell migration in situations of tissue remodeling in vivo.

In conclusion, secreted Jagged 1 can act at the level of vascular endothelium (cells expressing notch4) and induce the vascularization mechanism.

The autocrine and paracrine Jagged/Notch4 system in mast cells can contribute to angiogenesis. These results demonstrate that mast cells are effector cells of angiogenesis.

1. A method for treating tumor angiogenesis comprising administering a tyrosine kinase inhibitor to a mammalian in need of such treatment, wherein said inhibitor is unable to promote death of IL-3 dependent cells cultured in presence of IL-3.

2. A method according to claim 1, wherein said tyrosine kinase inhibitor is a non-toxic, selective and potent c-kit inhibitor.

3. A method according to claim 2, wherein said inhibitor is selected from the group consisting of indolinones, pyrimidine derivatives, pyrrolopyrimidine derivatives, quinazoline derivatives, quinoxaline derivatives, pyrazoles derivatives, bis monocyclic, bicyclic or heterocyclic aryl compounds, vinylene-azaindole derivatives and pyridyl-quinolones derivatives, styryl compounds, styryl-substituted pyridyl compounds, seleoindoles, selenides, tricyclic polyhydroxylic compounds and benzylphosphonic acid compounds.

4. A method for treating tumor angiogenesis comprising administering a non toxic, potent and selective c-kit inhibitor to a mammalian in need of such treatment, selected from the group consisting of:
pyrimidine derivatives, more particularly N-phenyl-2-pyrimidine-amine derivatives.
indolinone derivatives, more particularly pyrrol-substituted indolinones,
monocyclic, bicyclic aryl and heteroaryl compounds,
and quinazoline derivatives,
wherein said inhibitor is unable to promote death of IL-3 dependent cells cultured in presence of IL-3.

5. A method according to claim 4, wherein said inhibitor is a N-phenyl-2-pyrimidine-amine derivative selected from the compounds corresponding to formula II: 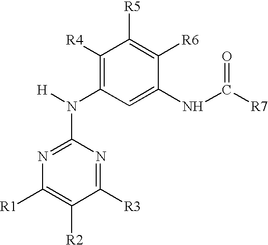
Wherein R1, R2 and R3 are independently chosen from H, F, Cl, Br, I, a C1-C5 alkyl or a cyclic or heterocyclic group, especially a pyridyl group;
R4, R5 and R6 are independently chosen from H, F, Cl, Br, I, a C1-C5 alkyl, especially a methyl group;
and R7 is a phenyl group bearing at least one substituent, which in turn possesses at least one basic site, such as an amino function.

6. A method according to claim 5, wherein R1 is a heterocyclic group, especially a pyridyl group, R2 and R3 are H, R4 is a C1-C3 alkyl, especially a methyl group, R5 and R6 are H, and R7 is a phenyl group bearing at least one substituent, which in turn possesses at least one basic site, such as an amino function.

7. A method according to claim 6, wherein R7 is the following group:

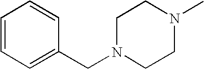

8. A method according to claim 4, wherein said inhibitor is the 4-(4-méhylpipérazine-1-ylméthyl)-N-[4-méthyl-3-(4-pyridine-3-yl)pyrimidine2 ylamino)phényl]-benzamide.

9. A method according to claim 4, wherein said inhibitor is an inhibitor of activated c-kit selected from a constitutively activated-mutant c-kit and/or SCF-activated c-kit.

10. A method according to claim 9, wherein the activated-mutant c-kit has at least one mutation selected from mutations proximal to Y823, more particularly between amino acids 800 to 850 of SEQ ID No1 involved in c-kit autophosphorylation, notably the D816V, D816Y, D816F and D820G mutants, and a deletion in the juxtamembrane domain of c-kit, preferably between codon 573 and 579.

11. A method for treating tumor angiogenesis comprising administering to a mammalian in need of such treatment a compound that is a selective, potent and non toxic inhibitor of activated c-kit obtainable by a screening method which comprises:
a) bringing into contact (i) activated c-kit and (ii) at least one compound to be tested; under conditions allowing the components (i) and (ii) to form a complex,
b) selecting compounds that inhibit activated c-kit,
c) testing and selecting a subset of compounds identified in step b), which are unable to promote death of IL-3 dependent cells cultured in presence of IL-3.

12. A method according to claim 11, wherein the screening method further comprises the step consisting of testing and selecting a subset of compounds identified in step b) that are inhibitors of mutant activated c-kit, which are also capable of inhibiting SCF-activated c-kit wild.

13. A method according to claim 11, wherein activated c-kit is SCF-activated c-kit wild.

14. A method according to one of claims 11 to 13, wherein putative inhibitors are tested at a concentration above 10 μM in step a).

15. A method according to one of claims 11 to 14, wherein IL-3 is present in the culture media of IL-3 dependent cells at a concentration comprised between between 0.5 and 10 ng/ml, preferably between 1 to 5 ng/ml.

16. A method according to one of claims 11 to 15, wherein the extent to which component (ii) inhibits activated c-kit can be measured in vitro or in vivo.

17. A method according to one of claims 11 to 16 wherein the screening method further comprises the step consisting of testing and selecting in vitro or in vivo compounds capable of inhibiting c-kit wild at concentration below 1 μM.

18. A method according to claim 17 wherein, wherein the test is performed using cells lines selected from the group consisiting of mast cells, transfected mast cells, BaF3, and IC-2.

19. A method according to claim 17, wherein the test includes the determination of the amount of c-kit phosphorylation.

20. A method for treating tumor angiogenesis according to one of claims 11 to 18, wherein the screening comprises:
a) performing a proliferation assay with cells expressing a mutant c-kit (for example in the transphosphorylase domain), which mutant is a permanent activated c-kit, with a plurality of test compounds to identify a subset of candidate compounds targeting activated c-kit, each having an IC50<10 μM, by measuring the extent of cell death,
b) performing a proliferation assay with cells expressing c-kit wild said subset of candidate compounds identified in step (a), said cells being IL-3 dependent cells cultured in presence of IL-3, to identify a subset of candidate compounds targeting specifically c-kit,
c) performing a proliferation assay with cells expressing c-kit, with the subset of compounds identified in step b) and selecting a subset of candidate compounds targeting c-kit wild, each having an IC50<10 μM, preferably an IC50<1 μM, by measuring the extent of cell death.

21. A method according to one of claims 1 to 20 for treating tumor angiogenesis in human.

22. A method according to claim 21, wherein the inhibitor is administered orally.

23. A method according to claim 21, wherein the inhibitor is administered topically

24. Use of a non toxic, potent and selective c-kit inhibitor for preparing a medicament for treating tumor angiogenesis inhuman.


SEQUENCE LISTING (5 sequences)

SEQ ID NO: 1 - protein, 976 aa, Homo sapiens [sequence omitted]

SEQ ID NO: 2 - DNA, 30 bp, Homo sapiens
  Primer
aagaagagatggtacctcgaggggtgaccc

SEQ ID NO: 3 - DNA, 33 bp, Homo sapiens
  Primer
ctgcttcgcggccgcgttaactcttctcaacca

SEQ ID NO: 4 - DNA, 20 bp, Homo sapiens
  Primer
agctcgtttagtgaaccgtc

SEQ ID NO: 5 - DNA, 20 bp, Homo sapiens
  Primer
gtcagacaaaatgatgcaac